(19) United States
(12) Patent Application Publication
(10) Pub. No.: US 2006/0069155 A1
(43) Pub. Date: Mar. 30, 2006

(54) Method for reducing exacerbations associated with COPD

(76) Inventor: Jeffrey D. Edelson, Berwyn, PA (US)

Correspondence Address: GLAXOSMITHKLINE;Corporate Intellectual Property - UW2220, P.O. Box 1539, King of Prussia, PA, 19406-0939, US

(21) Appl. No.: 11/280,089

(22) Filed: Nov. 16, 2005

Related U.S. Application Data
(63) Continuation of application No. 10/856,460, filed on May 28, 2004, now abandoned.
Application No. 10/856,460 is a continuation of application No. 10/343,078, filed on Jan. 27, 2003, now abandoned.
(60) Provisional application No. 60/221,275, filed on Jul. 27, 2000.

(51) Int. Cl. A61K 31/277

(52) U.S. Cl. 514/521

(57) ABSTRACT

This invention relates to a method for reducing the incidences and/or the severity of exacerbations of COPD by administering a phosphodiesterase 4 (PDE4) inhibitor.

### CROSS-REFERENCE OF PREVIOUS APPLICATIONS

This application is a continuation of application Ser. No. 10/856,460 filed 28 May 2004 which is a continuation of application Ser. No. 10/343,078 filed 27 Jan. 2003 (now abandoned), which is a 371 national stage of PCT/US01/23542 filed Jul. 26, 2001, which claims benefit of U.S. Provisional Application No. 60/221,275, filed Jul. 27, 2000.

Scope

This invention relates to a method for reducing the incidences and/or the severity of exacerbations of COPD by administering a phosphodiesterase 4 (PDE4) inhibitor.

### BACKGROUND

Chronic obstructive pulmonary disease (COPD) is characterized by a reduction in expiratory flow and slow forced emptying of the lungs which does not change markedly over several months (1). The disease is primarily caused by smoking, has a high incidence of mortality and morbidity and is poorly served by existing therapies. In the UK, COPD accounts for approximately 6% of deaths in men and 4% of deaths in women and is the third most common cause of death (2). The World Health Organization Global Burden of Disease study showed COPD to be the sixth leading cause of death worldwide in 1990 and is predicted to rise to third position by 2020. COPD is associated with major healthcare costs, largely due to expensive treatments such as long-term oxygen therapy and hospital admissions, as well as indirect costs including loss of working capacity. Recent epidemiological data suggests that the prevalence of the disease is underestimated. Based on data from NHANES III (1988-1994) for subjects in the United States, it was estimated that 4.6% of men and 3.7% of women had a diagnosis of COPD and another 24.2% of men and 16.7% of women had airflow obstruction that was undiagnosed (4). Patients with COPD frequently develop acute exacerbations of the disease that are an important cause of morbidity and mortality and have a significant economic impact. On average, patients experience one or two exacerbations per year and the frequency increases as the disease progresses (Anthonisen NR, Manfreda J, Warran CPW et al, Antibiotic therapy in exacerbations of chronic obstructive pulmonary disease. Ann Intern. Med 1987, 106: 196-204; Fletcher C, Peto R. The natural history of chronic airflow obstruction. BMJ, 1977; 1: 1645-1648).

Cyclic nucleotide phosphodiesterases (PDEs) represent a family of enzymes that hydrolyze the ubiquitous intracellular second messengers, adenosine 3′,5′-monophosphate (cAMP) and guanosine 3′,5′-monophosphate (cGMP) to their corresponding inactive 5′-monophosphate metabolites. At least seven distinct classes of PDE isozymes are believed to exist, each possessing unique physical and kinetic characteristics and each representing a product of a different gene family. These are distinguished using Arabic numerals 1-7.

The target enzyme for use of the formulations of this invention is the PDE 4 isozyme in all its various forms and in the full domain of its distributions in all cells. It is a low Kₘ (cAMP Kₘ=1-5 μM) cAMP-selective enzyme that has little activity against cGMP (Km>100 μM). Members of this isozyme class have the interesting characteristics of existing in two or more non-interconvertible or slowly interconvertible forms that bind rolipram and other PDE IV inhibitors with distinct rank-order potencies. Thus the same gene product can exist in more than one catalytically active conformational state. Importantly, the relative proportions of the different binding forms may vary depending on the tissue cell type. For example, inflammatory cells may contain a relatively high proportion of the form that binds rolipram with a low affinity while brain and parietal cells may contain a relatively high proportion of the form that binds rolipram with a high affinity. Current PDE inhibitors used in treating inflammation and as bronchodilators, drugs like theophylline and pentoxyfyllin, inhibit PDE isozymes indiscriminately in all tissues. These compounds exhibit side effects, apparently because they non-selectively inhibit all PDE isozyme classes in all tissues. The targeted disease state may be effectively treated by such compounds, but unwanted secondary effects may be exhibited which, if they could be avoided or minimized, would increase the overall therapeutic effect of this approach to treating certain disease states. Although in theory isozyme-selective PDE inhibitors should represent an improvement over non-selective inhibitors, the selective inhibitors tested to date are not devoid of side effects produced as an extension of inhibiting the isozyme of interest in an inappropriate or untargeted tissue. For example, clinical studies with the selective PDE 4 inhibitor rolipram, which was being developed as an antidepressant, indicate it has psychotropic activity and produces gastrointestinal effects, e.g., pyrosis, nausea and emesis. Indications are that side effects of denbufylline, another PDE 4 inhibitor targeted for the treatment of multi-infarct dementia, may include pyrosis, nausea and emesis as well. These side effects are thought to occur as a result of inhibiting PDE 4 in specific areas of the CNS and gastrointestinal system.

But it has been found that certain compounds which potently compete for the high affinity rolipram binding form (HPDE 4) have more side effects or more intense side effects than those which more potently compete with the LPDE 4 (low affinity rolipram binding form). Data is now available which indicate that compounds can be targeted to the low affinity binding form of PDE 4 and that this form is distinct from the binding form for which rolipram is a high affinity binder. Distinct SARs have been found to exist for inhibitors acting at the high affinity rolipram binding form versus the low affinity rolipram binding form. In addition, these two forms appear to have different functional roles. Thus compounds that interacted with the low affinity rolipram binding form appears to have anti-inflammatory activity, whereas those that interact with the high affinity rolipram binding form produce side effects or exhibit more intensely those side effects.

A useful consequence of these findings is that it is now possible to identify compounds which preferentially inhibit cAMP catalytic activity where the enzyme is in the form that binds rolipram with a low affinity, thereby reducing the side effects which apparently are linked to inhibiting the form which binds rolipram with a high affinity. This provides a superior therapeutic index vis-a-vis anti-inflammatory and/or bronchodilator activities versus side effects. It has now been found that certain of these inhibitors, ones which do not induce unacceptable untoward adverse events when administered at dosages which treat COPD per se, will also when given at the same or smaller doses, reduce the incidences and/or severity of exacerbations of the disease that oft times affect COPD suffers.

### SUMMARY OF THE INVENTION

In a first aspect, this invention relates to a method for reducing the incidences and/or severity of exacerbations of COPD in a mammal suffering from COPD, the method comprising administering an effective amount of a PDE4 inhibitor to a patient whom is suffering from COPD.

In a further aspect, this invention relates to the use of a PDE4 inhibitor in the manufacture of a medicament for reducing the incidences and/or severity of exacerbations of COPD.

### DESCRIPTION OF THE FIGURES

FIG. 1: Kaplan-Meier Estimates of Percentage of Patients Exacerbation-free-ITT in Clinical Study A

FIG. 2: Kaplan-Meier Estimates of Percentage of Patients Exacerbation-free-ITT in Clinical Study B

FIG. 3: Kaplan-Meier Estimates of Percentage of Patients Exacerbation-free-ITT in Clinical Study C

FIG. 4: Relative Risk (95% CI) of a COPD Exacerbation in Principal Studies.

### DETAILED DESCRIPTION OF THE INVENTION

Acute COPD exacerbations, defined as worsening of COPD symptoms that required changes in treatment including antimicrobial therapy, a short course of oral corticosteroids or other bronchodilator therapy. Exacerbations were categorized to three levels:
Level 1: self-managed by the patient at home by increasing usual medication for COPD.
Level 2: requiring additional treatment prescribed by a family or primary care physician or as a result of a hospital outpatient visit including a visit to the Emergency Room.
Level 3: requiring the patient to be admitted to the hospital for treatment.

In certain clinicial studies A and B, patients who received Ariflo® (cis-4-cyano-4-[3-(cyclopentyloxy)-4-methoxyphenyl]cyclohexane-1-carboxylic acid), 15 mg immediate release tablet, BID, had a lower incidence of all categories of exacerbations as well as the more severe exacerbations (Levels 2 and 3) requiring treatment by a physician or hospitalization than patients who received placebo.

Clinical trials in COPD patients were carried out to investigate the efficacy of an 15 mg immediate release tablet, BID of Ariflo. These were placebo-controlled, parallel-group, double-blind studies of 6 months duration designed to demonstrate the efficacy and safety of Ariflo 15 mg BID in the treatment of COPD.

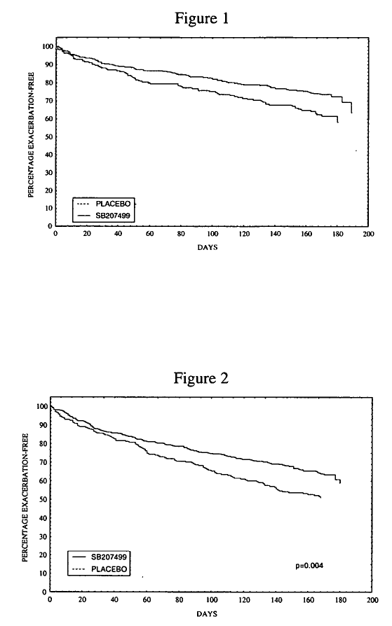

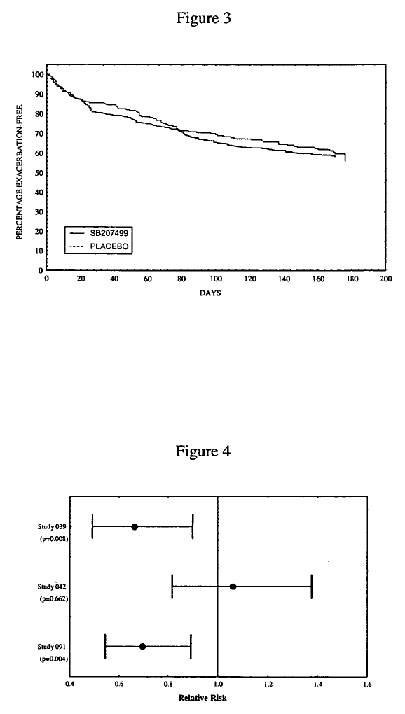

In one clinical study, A, treatment with Ariflo (15 mg immediate release tablet, BID) reduced the risk of having an exacerbation by 39% relative to placebo (P=0.002) and reduced the risk of an exacerbation requiring treatment by a physician or hospitalization by 45% (P=0.001) based on Kaplan-Meier estimates (FIG. 1). Similarly, in as second study B, treatment with Ariflo over 26 weeks reduced the risk of having an exacerbation by 30% relative to placebo (P=0.005) and reduced the risk of an exacerbation requiring treatment by a physician or hospitalization (Levels 2 and 3) by 32% (P=0.004) (FIG. 2). A third clinical trial C with Ariflo, 15 mg BID, over 6 months gave equivocal results as regards efficacy in treating COPD. In this study the affect of Ariflo on exacerbations was marginal (FIG. 3).

Exacerbation-free survival rates based on Kaplan-Meier estimates of time-to-first exacerbation are summarized in Table 1.

TABLE 1
Summary of Exacerbation-free Survival at 24 Weeks By Study

| | | | Exacerbation-free Survival Rate* | | | |
|---|---|---|---|---|---|---|
| | | Number of | Estimated | | | |
| | Total | Patients | Percentage | | | |
| Treatment | Number of | Exacerbation- | Exacerbation- | Lower | Upper | |
| Group | Patients | free | Free | 95% CI (%) | 95% CI (%) | P-value |

Study A
All Exacerbations

| | | | | | | |
|---|---|---|---|---|---|---|
| Placebo | 216 | 141 | 62.4 | 55.5 | 69.3 | |
| SB207499 | 431 | 336 | 74.0 | 69.3 | 78.6 | 0.008 |

Level 2 and 3 Exacerbations

| | | | | | |
|---|---|---|---|---|---|
| Placebo | 157 | 69.7 | 63.1 | 76.3 | |
| SB207499 | 364 | 81.7 | 77.5 | 85.8 | 0.003 |

Study C
All Exacerbations

| | | | | | | |
|---|---|---|---|---|---|---|
| Placebo | 226 | 143 | 56.0 | 47.8 | 64.2 | |
| SB207499 | 474 | 299 | 58.3 | 53.5 | 63.1 | 0.662 |

Level 2 and 3 Exacerbations

| | | | | | |
|---|---|---|---|---|---|
| Placebo | 164 | 71.0 | 64.7 | 77.3 | |
| SB207499 | 354 | 70.9 | 66.5 | 75.3 | 0.793 |

Study B
All Exacerbations

| | | | | | | |
|---|---|---|---|---|---|---|
| Placebo | 242 | 135 | 51.1 | 44.4 | 57.8 | |
| SB207499 | 469 | 318 | 63.9 | 59.1 | 68.6 | 0.004 |

Level 2 and 3 Exacerbations

| | | | | | |
|---|---|---|---|---|---|
| Placebo | 165 | 64.3 | 57.9 | 70.8 | |
| SB207499 | 367 | 75.5 | 71.2 | 79.7 | 0.009 |

Results for the intent-to-treat population are presented.
*Exacerbation-free survival rate is estimated using Kaplan-Meier estimates of time-to-first exacerbation. P-values based on log-rank test.

In the two clinical studies A and B where Ariflo (15 mg IR, BID) showed efficacy in treating COPD, the estimated percentages of patients exacerbation-free were significantly greater for patients who received a 15 mg IR tablet BID of Ariflo compared to patients who received placebo (P=0.008 and P=0.004, respectively) (FIG. 4).

The benefit of Ariflo in reducing the risk of COPD exacerbations in two studies is an important clinical finding, since exacerbations are associated with a poor long-term clinical outcome and have significant implications for healthcare costs.

A preferred group of inhibitors are those that have an IC₅₀ ratio (high/low binding) of about 0.1 or greater, as that IC₅₀ ratio determination is described in U.S. Pat. No. 5,998,428. It is incorporated herein in full by reference as if fully set forth herein. A preferred standard for PDE 4-specific inhibitors which can be used in this invention is one where the compound has an IC₅₀ ratio of about 0.1 or greater; said ratio being the ratio of the IC₅₀ value for competing with the binding of 1 nM of [³H]R-rolipram to a form of PDE 4 which binds rolipram with a high affinity over the IC₅₀ value for inhibiting the PDE 4 catalytic activity of a form which binds rolipram with a low affinity using 1 uM[³H]-cAMP as the substrate.

Specific PDE 4 inhibitors that may be included in these formulations include those set out in U.S. Pat. No. 5,552,438 issued 03 Sep. 1996. This patent and the compounds it discloses are incorporated herein in full by reference. The compound of particular interest, which is disclosed in U.S. Pat. No. 5,552,438, is cis-4-cyano-4-[3-(cyclopentyloxy)-4-methoxyphenyl]cyclohexane-1-carboxylic acid and its salts, esters, pro-drugs or physical forms. This compound is identified here by its IUPAC name, by its registered trademark Ariflo, by its generic name cilomilast, and by an alphanumeric SB207499. Other PDE 4 inhibitors which may be of interest include: AWD-12-281 from Astra (Hofgen, N. et al. 15th EFMC Int Symp Med Chem (Sep. 6-10, Edinburgh) 1998, Abst P.98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (Sep. 19-23, Geneva) 1998] 1998, 12(Suppl. 28): Abst P2393); roflumilast (CAS reference No 162401-32-3) and a pthalazinone (WO 9947505) from Byk-Gulden; and a compound identified as T-440 (Tanabe Seiyaku; Fujii, K. et al. J Pharmacol Exp Ther, 1998, 284(1): 162). Preferred compounds of this invention are those which have an IC₅₀ ratio of greater than 0.5, and particularly those compounds having a ratio of greater than 1.0. The most preferred compounds are roflumilast and cis-4-cyano-4-[3-(cyclopentyloxy)-4-methoxyphenyl]cyclohexane-1-carboxylic acid.

Other drugs useful in treating PDE4-related diseases can be incorporated into this therapy as well. Examples of other therapeutics by category, are drugs which treat: inflammatory respiratory diseases such as bronchodilators, leukotriene receptor antagonists and leukotriene biosynthesis inhibitors; non-respiratory inflammatory diseases such as irritable bowel disease (IBD); immunomodulating drugs, cognition enhancers; drugs for treating rheumatoid arthritis, rheumatoid spondylitis, osteoarthritis, gouty arthritis and other arthritic conditions; sepsis; septic shock; endotoxic shock; gram negative sepsis; toxic shock syndrome; adult respiratory distress syndrome; cerebral malaria; silicosis; pulmonary sarcoidosis; drugs for treating bone resorption diseases; reperfusion injury; graft vs. host reaction; allograft rejections; fever and myalgias due to infection, such as influenza, cachexia secondary to infection or malignancy, cachexia secondary to human acquired immune deficiency syndrome (AIDS), AIDS, ARC (AIDS related complex); keloid formation; scar tissue formation; Crohn's disease; ulcerative colitis; pyresis; autoimmune diseases such as multiple sclerosis, autoimmune diabetes and systemic lupus erythematosis; drugs for treating viral infections such as cytomegalovirus (CMV), influenza virus, adenovirus, and the herpes virus, and drugs for treating yeast and fungal infections.

Exemplary types of compounds for treating respiratory diseases are leukotriene antagonists; mucolytics; antitussives and expectorants; antibiotics; oral or inhaled beta-agonists; phosphodiesterase inhibitors other that PDE4-specific inhibitors; nasal decongestants; elastase inhibitors; protein therapeutics such as IL4, IL5, IL8, and IL13 monoclonal antibodies, anti-IgE; or oral or inhaled corticosteriods. Particularly preferred combination therapies are the use of a therapeutic amount of a corticosteriod, a beta agonist, an anticholinergic, an inhaled cromone, a leukotriene antagonist, or an antibiotic to treat secondary infections.

The amount of inhibitor that is effective in this treatment method falls between 100 micrograms and 100 mg per dose, administered as needed from one to four times per day. A preferred range is 1-60 mg per dose administered once or twice a day. More preferred is a 5-30 mg dose administered one or twice a day. Most preferred is a 10-20, or 10-15 mg dose administered once or twice per day, e.g. a twice-a-day 15 mg dose, or once-a-day 30 or 60 mg dose. The dose for reducing exacerbations and/or the severity of them can be smaller than that which is used to treat COPD per se.

The inhibitor will be administered by conventional means. For example, it will be administered orally or as an inhaled powder or aerosol. It may be possible to formulate some of these inhibitors in the form of a topical patch, a sustained release injectable or a suppository, it is believed that an oral preparation or one administered as an inhalant will be the superior route of delivery.

For the purposes of this invention, the preferred formulation will be an immediate release or controlled release oral tablet containing between about 1 mg to 200 mg of Ariflo, more preferably 5 to 100 mg, and most preferably between 5, or 10 to 60 mg of the active ingredient. Additional preferred dosage amounts within these ranges are 10, 15, 20, 30, 40, 50, 60, 70, 80 or 90 mg per preparation.

### SPECIFIC EXAMPLES

The clinical studies protocol were carried out generally as follows:

Eligible patients had a clinical diagnosis of COPD (according to international treatment guidelines), a % predicted FEV₁≧30% and ≦70% post-bronchodilator, a FEV₁/FVC of ≦0.7, and fixed airway obstruction defined by ≦15% reversibility following administration of a beta₂-agonist. In each of the studies, patients with COPD entered a 4-week placebo run-in period and were then randomized to receive Ariflo 15 mg twice daily or placebo in a ratio of 2 to 1. Patients were monitored following 1, 2 and 4 weeks of treatment and subsequently at 4-week intervals. Patients were permitted to receive concomitant salbutamol (prn) and/or short acting anticholinergic therapy at a stable dose during these studies.

Study B included a 2-week, randomised, double-blind, run-out phase to examine the effects of discontinuation of treatment. Patients who received Ariflo during the initial 24 week period, were randomized (1:1 ratio) to Ariflo 15 mg BID or placebo for the run-out phase; patients who received placebo during the initial 24 weeks, continued on placebo during the run-out phase.

Pulmonary function measurements were performed at each visit (with the exception of Week 1). The St. George's Respiratory Questionnaire (SGRQ) was administered at Baseline, and Weeks 12 and 24 (or at the time of withdrawal). Routine compliance, vital sign, and laboratory assessments were performed at each visit. These studies included frequent ECG assessments and extensive monitoring of patients reporting gastrointestinal adverse experiences of potential clinical concern to address concerns about possible effects of SB 207499 on both cardiovascular and gastrointestinal body systems. 12-lead ECG assessments were conducted at screening, prior to dosing at Baseline, and prior to dosing at each visit during the double-blind treatment period. In addition, 12-lead ECGs were performed 3 hours after the administration of drug on the first and last day of dosing. In a subset of patients, 24-hour Holter ECG monitoring was conducted during Run-in and at Weeks 1 and 20. Orthostatic vital signs and faecal occult blood tests were determined for all patients in order to obtain a general incidence of abnormal assessments in this patient population. Orthostatic vital signs were determined at Screening, Baseline and the end of double-blind treatment. Faecal occult blood tests were performed between Screening and Baseline and between 20 weeks and the end of double-blind treatment (24 weeks).

All efficacy measures were analyzed for the intent-to-treat population (ITT), defined as all patients who received randomized study medication and had a baseline evaluation and at least one on-therapy efficacy evaluation during the double-blind period. Efficacy analyses for the per protocol population (PP) (defined as patients who did not significantly violate the protocol) were limited to trough FEV₁, and the total score of the SGRQ, the co-primary measures of efficacy, and the secondary parameters, exercise tolerance test, post-exercise breathlessness, summary symptom score, and trough FVC.

What is claimed is:

1. A method for reducing the incidences and/or severity of exacerbations of COPD in a mammal suffering from COPD, the method comprising administering an effective amount of a PDE4 inhibitor to a patient whom is suffering from COPD.

2. The use of a PDE4 inhibitor in the manufacture of a medicament for reducing the incidences and/or severity of exacerbations of COPD.
